(19) United States
(12) Patent Application Publication
(10) Pub. No.: US 2007/0239261 A1
(43) Pub. Date: Oct. 11, 2007

(54) Aneurysm occlusion system and method

(76) Inventors: Arani Bose, New York, NY (US); David Barry, Livermore, CA (US); Vikas Gupta, San Leandro, CA (US); Aleksandr Leynov, Walnutt Creek, CA (US); Delilah Hui, American Canyon, CA (US)

Correspondence Address: STALLMAN & POLLOCK LLP, 353 SACRAMENTO STREET, SUITE 2200, SAN FRANCISCO, CA, 94111, US

(21) Appl. No.: 11/784,236

(22) Filed: Apr. 6, 2007

Related U.S. Application Data
(60) Provisional application No. 60/790,160, filed on Apr. 7, 2006.

(51) Int. Cl. A61F 2/06

(52) U.S. Cl. 623/1.15

(57) ABSTRACT

An aneurysm occlusion device is positionable within a cerebral blood vessel covering a neck of an aneurysm on the blood vessel. The device includes a tubular element having a lumen surrounded by an occlusive sidewall including a plurality of gaps. The gaps are sufficiently small to cause at least partial occlusion against flow of blood from the blood vessel through the side wall into the aneurysm, but are expandable in response to a fluid pressure differential between a first area inside the lumen and a second area outside the lumen to allow flow of fluid through the side wall between the blood vessel and a side branch vessel.

### PRIORITY CLAIM

This application claims the priority of U.S. Provisional Application Ser. No. 60/790,160, filed Apr. 7, 2006.

### TECHNICAL FIELD OF THE INVENTION

The present invention relates generally to the field of aneurysm treatment and more particularly to a system and method for endovascular treatment of aneurysms.

### BACKGROUND

An aneurysm is an abnormal ballooning of a region of an artery wall caused by a weakening of the wall tissue.

While aneurysms can occur in any artery of the body, a large percentage of aneurysms are found in the cerebral blood vessels. If left untreated, such aneurysms can rupture, leading to life threatening hemorrhaging in the brain which can result in death or severe deficit. Aneurysms that do not rupture can form blood clots which can break away from the aneurysm potentially causing a stroke. In some patients, aneurysm can put pressure on nerves or brain tissue, causing pain, abnormal sensations, and/or seizures.

One current practice for treatment of an aneurysm includes surgical placement of an aneurysm clip across the aneurysm to prevent blood flow into the aneurysm. Naturally, this procedure requires highly invasive brain surgery and thus carries many risks.

In a less invasive catheter-based technique for aneurysm treatment, filler material is carried through the vasculature to the site of the aneurysm and used to pack the aneurysm. Materials used for this purpose include platinum coils and cellulose acetate polymer to fill the aneurysm sac. While these techniques have had some success, questions remain concerning their long-term effectiveness, ease of use, as well as their potential for rupturing the aneurysm or triggering clot formation.

According to another prior art aneurysm treatment, a mesh or braided stent-like device is positioned within a blood vessel such that it bridges the aneurysm, blocking flow of blood into the aneurysm. A problem encountered with devices of this type is that the sidewalls of the devices not only occlude blood flow into the aneurysm, but they will also block blood flow between the blood vessel and any side branch vessels that the stent happens to cover. See FIG. 1 which shows a blood vessel V, aneurysm A, and side branch vessel B. In some prior art modifications to the stent-type devices, the devices include sidewalls that are not occlusive around the full circumference of the device. In implanting these devices, the physician must make certain that the occlusive portion of the device's circumference covers the aneurysm and not any of the side branch vessels.

The present application describes aneurysm occlusion devices that are effective at occluding blood flow into aneurysms without impairing blood flow into or from side branch vessels.

### BRIEF DESCRIPTION OF THE DRAWINGS

FIG. 1 schematically illustrates an aneurysm in a blood vessel and the corresponding blood flow.

FIG. 2A is a side elevation view of the components of an aneurysm occlusion system.

FIG. 2B is a side elevation view of the system of FIG. 2A, showing the components assembled for use.

FIGS. 3A-3F are plan views of various embodiments of occlusion devices for the system of FIG. 2A. Although the occlusion devices are preferably tubular structures, each of FIGS. 3A-3F the device opened as if it was longitudinally cut and flattened into a sheet so that its features may be more easily viewed.

FIGS. 4A and 4B are perspective views of the occlusion device of FIG. 3A.

FIG. 5A is a plan view similar to FIG. 3A of another alternative occlusion device.

FIG. 5B is a perspective view of the occlusion device of FIG. 5A.

FIG. 6 is a plan view similar to FIG. 3A of still another alternative occlusion device.

FIG. 7A is a plan view similar to FIG. 3A of another alternative occlusion device before the device is shape set into a helical form.

FIG. 7B is a view similar to FIG. 7A showing the device after it has been shape set to include a right hand twist.

FIG. 7C is a view similar to FIG. 7A showing the device after it has been shape set to include a left hand twist.

FIG. 7D is a perspective view of the central portion of the device of FIG. 7B.

FIG. 8 is a plan view similar to FIG. 3A of another alternative occlusion device.

FIG. 9 is a plan view similar to FIG. 3A of another alternative occlusion device.

FIG. 10 is a plan view similar to FIG. 3A showing the devices of FIGS. 7B and 7C positioned overlapping one another.

FIG. 11 is a perspective view showing the central portion of the overlapping devices of FIG. 10.

FIG. 12A is a plan view similar to FIG. 3A showing another embodiment of an occlusion device; the device is shown positioned in a re-sheathable orientation.

FIG. 12B illustrates the device of FIG. 12A positioned in a non-resheathable orientation.

FIG. 12C illustrates a pair of the devices of 12A positioned in an overlapping arrangement, with an outer device positioned as oriented in FIG. 12B, and an inner device positioned as oriented in FIG. 12A.

FIGS. 13A-13E are a series of drawings schematically illustrating an aneurysm in a blood vessel, and showing a sequence of steps for deploying the aneurysm occlusion system of FIG. 1.

FIG. 14 is a perspective view of an alternative embodiment of an aneurysm occlusion device suitable for bifurcated vessels.

FIG. 15 is a side elevation view of the aneurysm occlusion device of FIG. 14.

FIG. 16 schematically illustrates a bifurcated vessel having an aneurysm, and shows the aneurysm occlusion device of FIG. 14 within the vessel.

FIG. 17A is a plan view illustrating the pattern used to cut the aneurysm occlusion device of FIG. 14 from a tube. Although the pattern is generally cylindrical, for simplicity FIG. 17A shows the pattern as if it were longitudinally cut and flattened.

FIG. 17B is a perspective view showing tubing following cutting using the pattern of FIG. 17A to form the occlusion device of FIG. 14, but prior to the step of shape setting the device into its final shape.

FIGS. 18A-18F are a sequence of drawings illustrating implantation of the occlusion device of FIG. 14.

### DETAILED DESCRIPTION

An embodiment of an aneurysm occlusion system 100 is shown in FIG. 2A. Generally speaking, system 100 includes an occlusion device 10, a sheath 12, and a pusher 14. A guidewire 16 may also be used with the system 100.

The occlusion device 10 is a tubular device capable of being retained in a constrained form or shape prior to deployment, and then expanded into contact with the walls of a vessel when deployed. Suitable materials for the sleeve include shape memory materials including superelastic Nitinol or shape memory polymers, or other materials such as stainless steel, composite materials, or combinations of metals and polymeric materials. In a preferred embodiment, the occlusion device 10 may be formed by laser cutting features into a length of superelastic Nitinol tubing, and then chemically processing and shape-setting the material one or more times using methods known to those skilled in the art. As will be discussed in greater detail below, the walls of the device 10 are constructed to restrict passage of blood from a vessel into an aneurysm protruding from that vessel, without compromising blood flow into any side branch vessels that might be present in the region of the aneurysm.

The occlusion device 10 is proportioned to be implanted within the cerebral vasculature including, but not limited to, the Internal Carotid Artery, External Carotid Artery, Vertebral Artery, Basilar Artery, Middle Cerebral Artery, Anterior Cerebral Artery, and the Posterior Cerebral Artery. Preferred devices 10 are expandable to an outer diameter in the range of 2.0 mm-6.0 mm. The user may be provided with a set of multiple occlusion devices of different diameters so that the device with the most appropriate dimensions may be chosen for the procedure.

Sheath 12 is an elongate tubular catheter preferably formed of a polymeric material such as Pebax, nylon, urethane, PTFE, Polyimide, metals such as Stainless Steel, Platinum etc., or other suitable materials. A central lumen 13 extends the length of the sheath 12. The sheath is proportioned for passage through cerebral vascular, and may have an outer diameter in the range of 1 mm-2 mm.

Pusher 14 is an elongate tubular member having a lumen 18. The distal end of the pusher 14 includes an atraumatic tip having a flared section 20 and a tapered section 22. A cylindrical shoulder 24 is positioned on the exterior of the pusher 14, at a location proximal to, and spaced apart from, the flared section 20. The pusher may be formed of suitable polymers, metals, and/or composite materials. Referring to FIG. 2B, when the system 100 is assembled for deployment of the occlusion device 10, the device 10 is threaded over the pusher 14, radially compressed to its constrained position, and positioned with its proximal end in abutment with the shoulder 24 on the exterior surface of the pusher 14. Sheath 12 is positioned over the pusher and the occlusion device 10 to maintain the device 10 in the constrained position as shown in FIG. 2B.

The distal end of the pusher 14 may include a hook (not shown) or equivalent mechanism detachably engaged with a proximal portion of the device 10. Where provided, the hook may be used for withdrawing the device 10 back into the sheath 12 if, after the device has been partially deployed, it is determined that a smaller or larger device should be used, or if the device needs to be repositioned. Once the device is finally deployed, the hook is detached from the device. Similar systems for resheathing and/or repositioning intravascular devices may be found in the intravascular stent art.

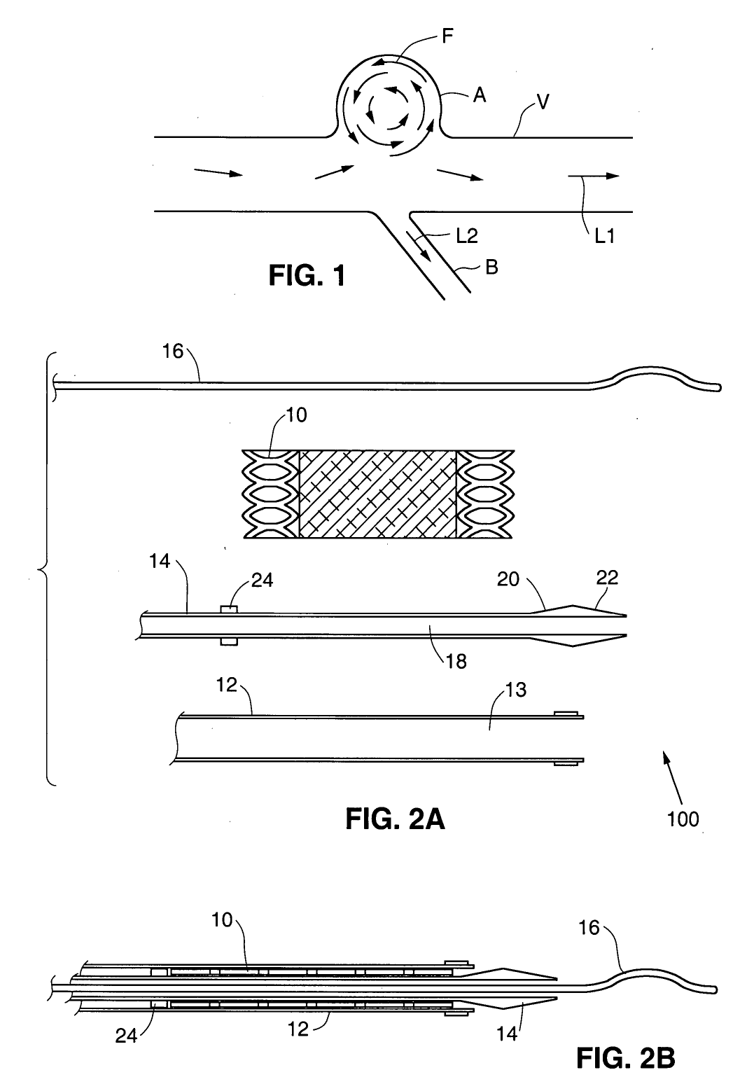

The occlusion device 10 can be configured in a number of ways. Referring to FIG. 1, a preferred occlusion device includes features such that, when the device is positioned within a blood vessel V covering the opening to an aneurysm A, it will occlude blood flow into the aneurysm without significantly blocking blood flow into branch vessel B, even if the position of the occlusion device covers the opening to the side branch vessel. Several embodiments of occlusion devices, each of which includes this preferred feature, are described herein. However, it should be appreciated that various other embodiments are conceivable without departing from the scope of the present invention.

The disclosed embodiments rely on the differences between the fluid dynamics at the location of the aneurysm and the fluid dynamics at the side branch vessel. The mean arterial pressure and flow characteristics within the circulatory system vary as a function of the distance from the heart, location, and vessel diameter. Flow is driven by normal pressure gradients, from the arterial side to the venous side of the circulatory system, except in circumstances of abnormal or physiologic arterio-venous shunting. Pressure and flow within the various compartments of a particular angio-architectural space is determined by these factors. In general, the pressure ranges from mean arterial pressure in the range of 25-100 mmHg, to no greater than approximately 15 mmHg on the venous side.

Referring again to FIG. 1, the flow dynamics and the pressure in the parent vessel V differs from that within the branch vessel B heading towards the capillary beds, and there is a pressure gradient between the parent vessel V and the branch vessel B. However, since the aneurysm lacks venous outflow, there is no pressure gradient between the parent vessel V and the aneurysm A. Thus, within the aneurismal dilation A of the parent vessel V there are vortices (indicated by arrows F) instead of laminar flow patterns L1, L2 of the type present in the parent vessel V and the branch vessel B.

Preferred occlusion devices take advantage of these differences to occlude flow to the aneurysm without occluding side branch vessel flow. These devices include an occlusive sidewall having a number of gaps or pores. The term “sidewall” is used loosely to refer to structure surrounding a lumen, and is not intended to suggest an impermeable structure. The occlusive sidewall is the high coverage portion of the sidewall that is positioned covering the aneurysm.

Because of the small dimensions of the gaps in the device, neointima (new layers of endothelial cells) forming on the device can contribute to the occlusive nature of the device by blocking some or all of the gaps. Also, due to the small size of the gaps, the surface tension of blood within the gaps can also enhance the occlusive nature of the device. When the occlusive sidewall covers a branch vessel B, the pressure differentials between the blood flowing in the branch vessel B and the parent vessel V will allow blood to flow through the side wall between the parent vessel and the branch vessel. In some instances, this may be because the pressure differential causes a deflection of the material surround the gaps (e.g. the bands). Deflection might be, for example, longitudinal or radial, and it might be pulsatile or constant. In some embodiments, this deflection can cause an expansion of the gaps from an occlusive size to a size that is sufficient to allow blood flow between the branch vessel B and the parent vessel V to proceed. Moreover, pulsatile deflection can disrupt the uniformity of blood surface tension across the gaps, and/or it can prevent neointima from forming on the portion of the device covering the branch vessel, in either case functioning to allow blood flow through the gaps of the occlusive sidewall into a branch vessel. In other instances, the pressure differential itself (rather than movement of the structure surrounding the gaps) may disrupt blood surface tension and/or neointima formation so as to allow blood flow through the occlusive sidewall.

On the other hand, since there is no appreciable pressure drop between the parent vessel V and the aneurysm A, that portion of the sidewall will occlude the aneurysm due to the lack of effective expansion of the gaps, and/or due to the blood surface tension across the gaps, and/or due to the presence of neointima in/on the gaps.

In the illustrated embodiments, the dynamic gaps take the form of spaces between bands of the material that form the device's sidewalls. It should be appreciated, however, that other mechanisms may be used to create these dynamic gaps without departing from the scope of the present invention. For example, the sidewalls may be formed of a material having pores that elastically stretch in response to the pressure differentials between the parent vessel and a side vessel.

Moreover, the disclosed embodiments are configured such that the arrangement of the gaps in the occlusive sidewall is functionally uniform around the circumference of the occlusive sidewall. In other words, the behavior of the dynamic gaps over the aneurysm is not dependent on which portion of the occlusive sidewall is positioned over the aneurysm or on which portion of the occlusive sidewall covers a branch vessel. Thus, with these embodiments, the physician need not be concerned with trying to cover the aneurysm with a particular area along the circumference of the occlusive sidewall (also referred to has the “high coverage area”).

In referencing the drawings, like numerals will be used to refer to features of the different embodiments that are similar to one another.

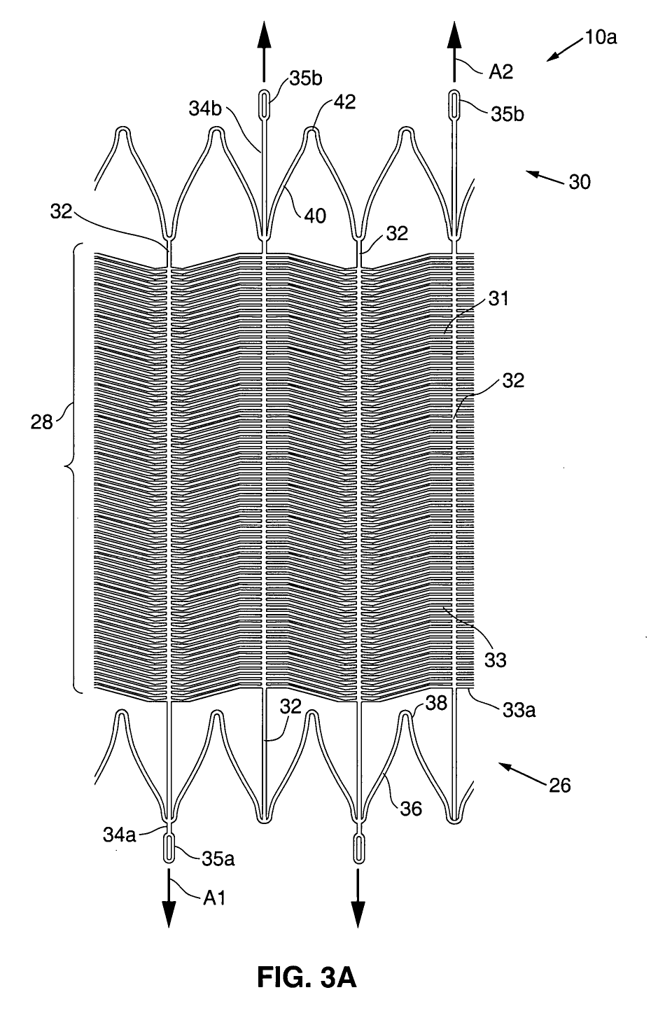

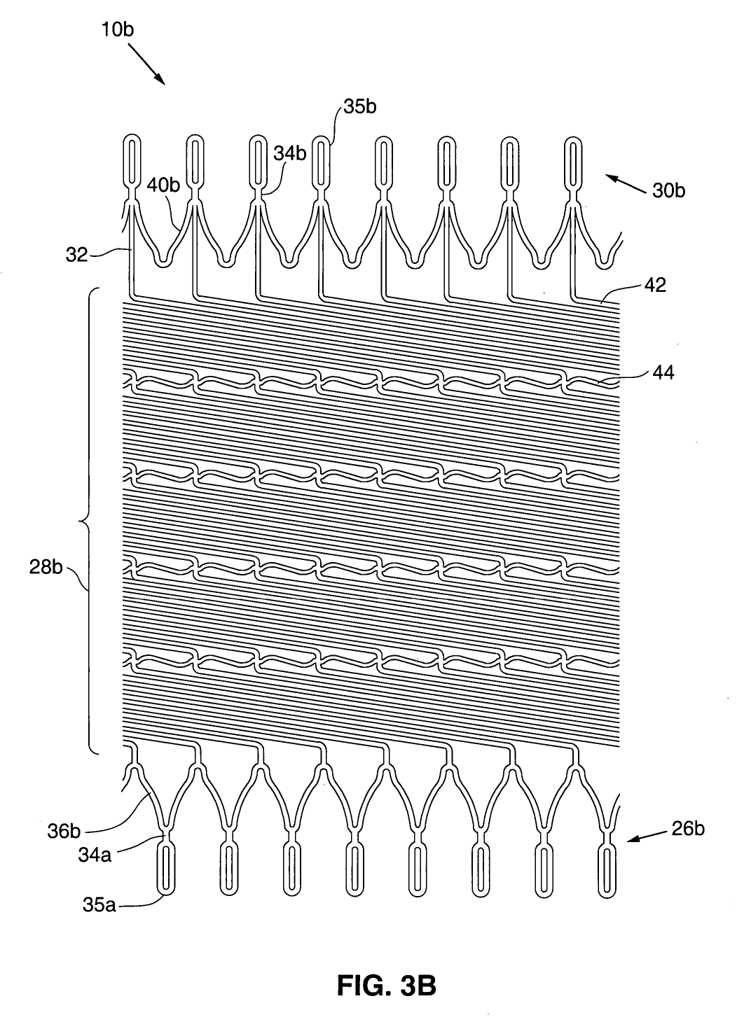

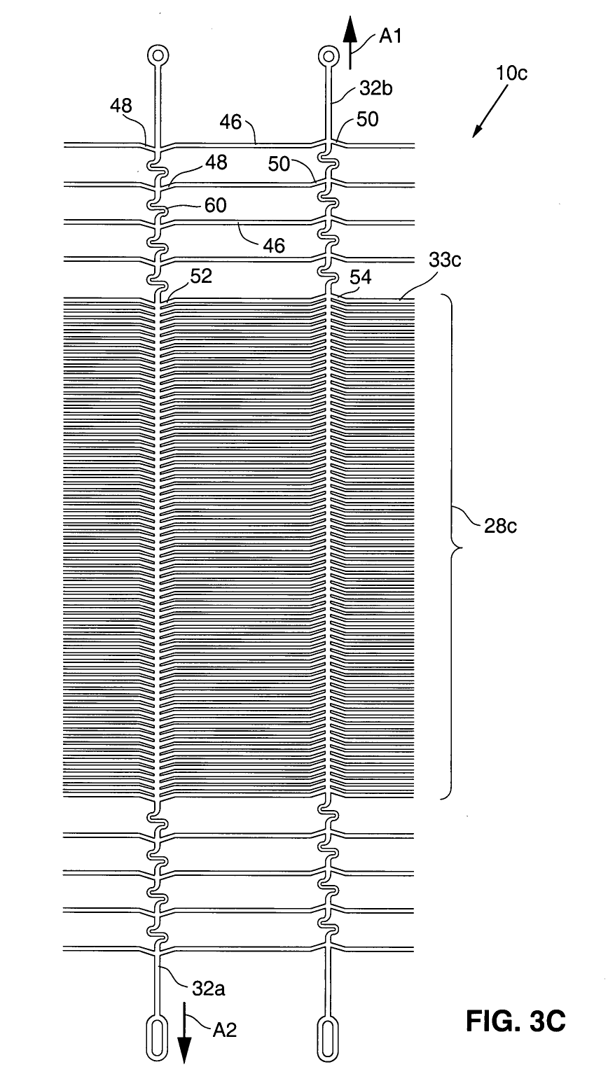

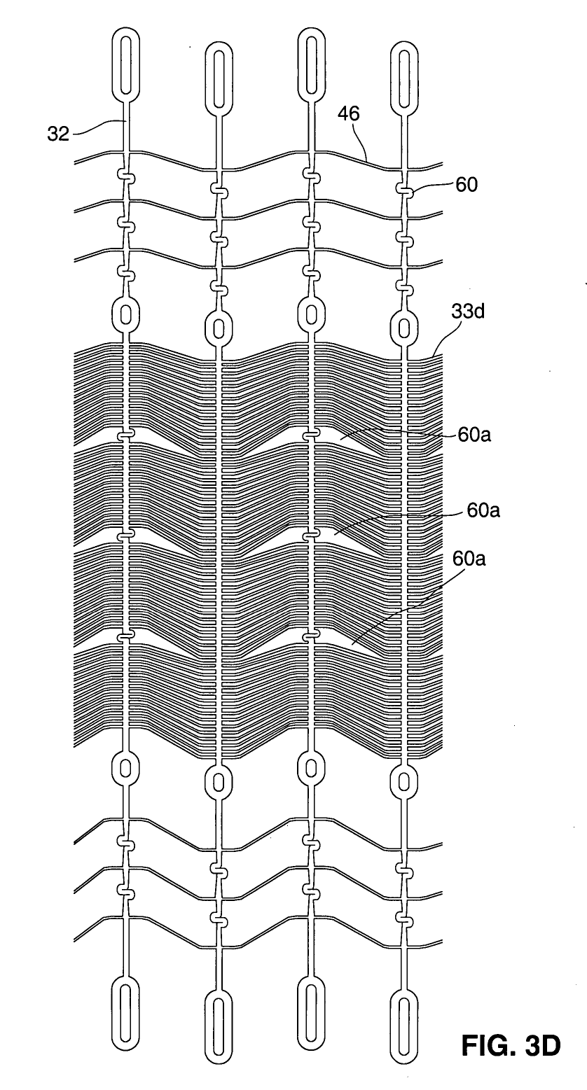

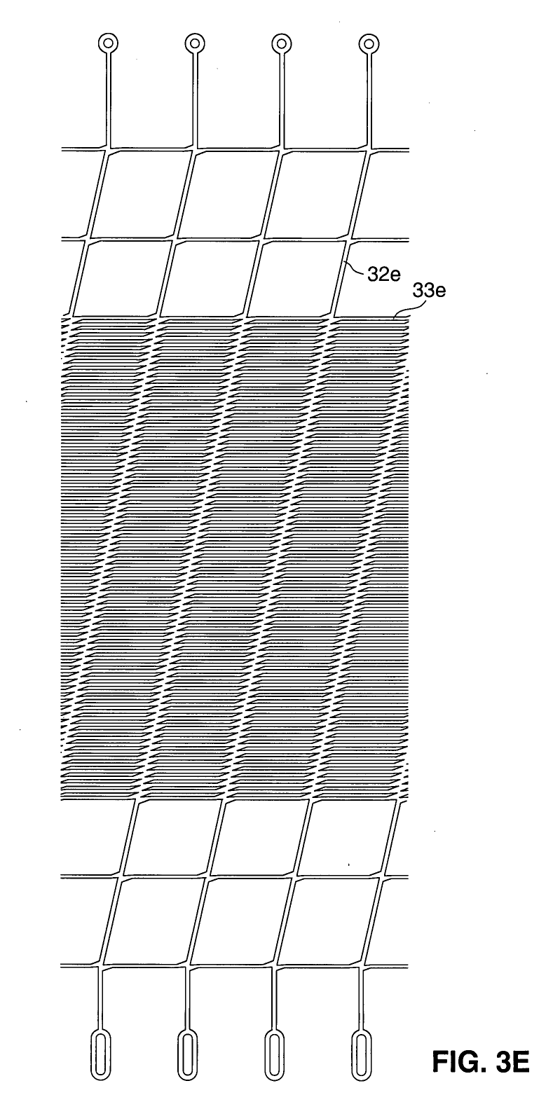

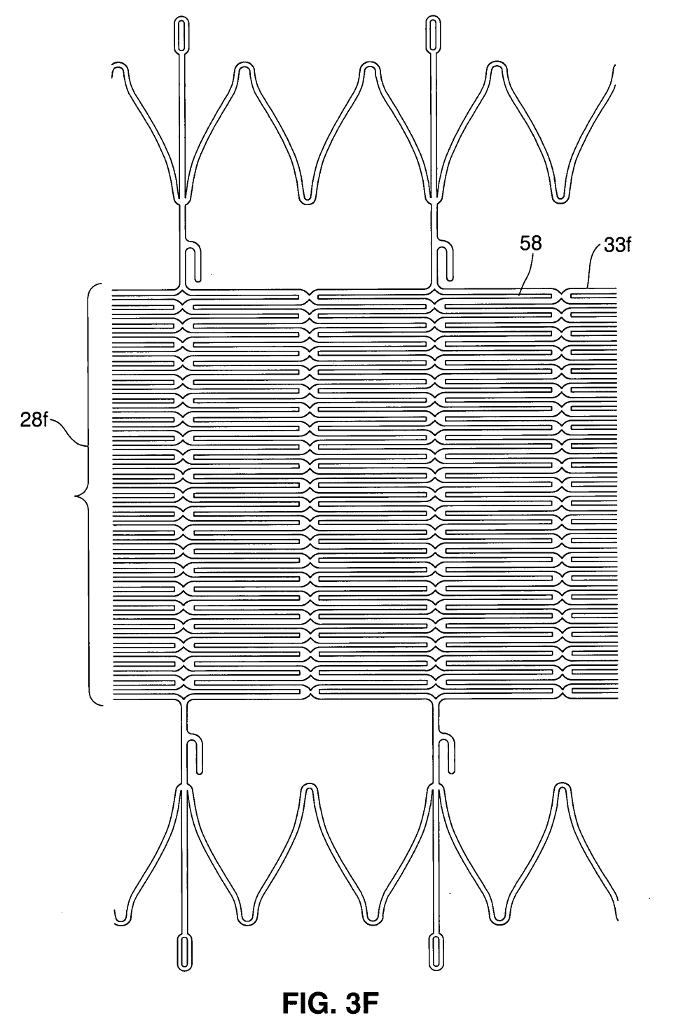

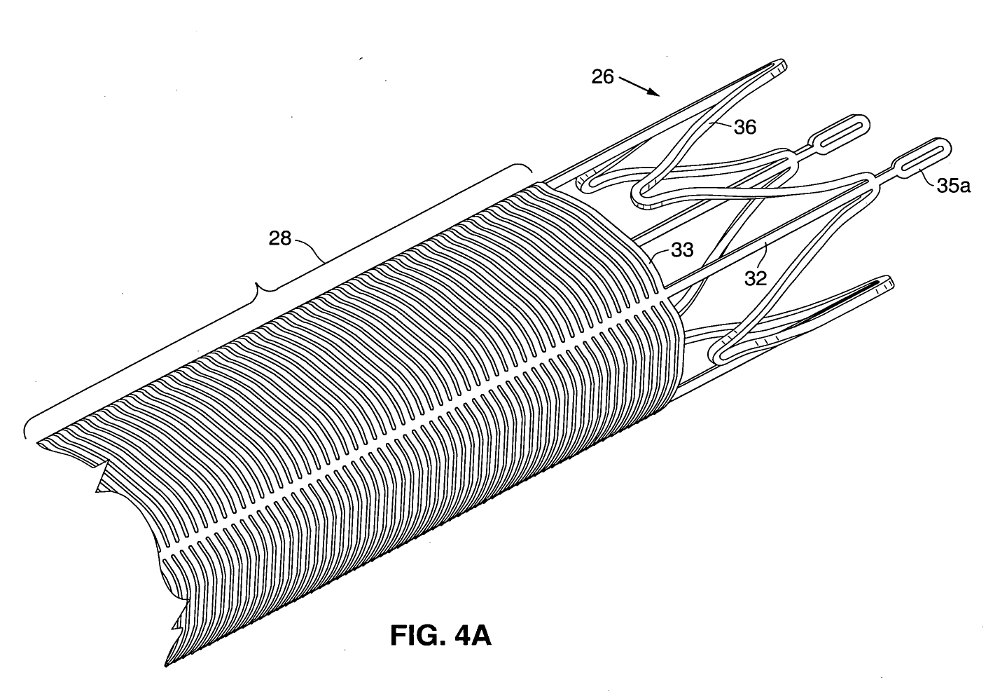

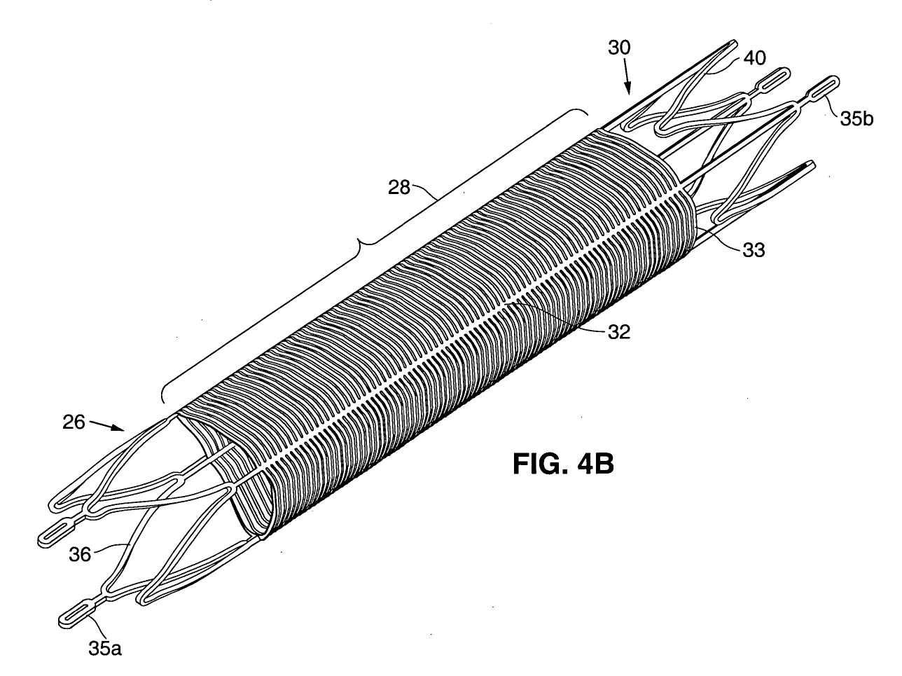

A first embodiment of an occlusion device 10a is shown in FIG. 3A. Occlusion device is preferably a tubular device having a proximal portion 26, a central portion 28, and a distal portion 30. The features of the device 10a are preferably formed by laser cutting features into a length of Nitinol tubing. The central portion 28, which is positioned to overlay the aneurysm during use, is cut into a high-coverage pattern having a plurality of gaps 31 separated by regions 33 of Nitinol material. As discussed above, the gaps 31 are arranged such that when a region of the central portion is positioned over a branch vessel B (FIG. 1), the fluid flow from the parent vessel V into the branch vessel B will separate the gaps by an amount sufficient to allow normal fluid flow into the branch vessel B. However, because there is minimal pressure differential between the parent vessel V and the aneurysm A, the gaps will not appreciably separate in the region of the central portion that is positioned over the aneurysm A. In this way, the central portion significantly reduces flow of blood into the aneurysm.

In the embodiment of FIG. 3A, the regions 33 take the form of a plurality of undulating cuffs, bands or ribbons defining the gaps 31. The curves or undulations in the cuffs, which may be near the points of intersection between the cuffs and elongate standards or uprights 32 (see description below), help allow the device to fold into a compressed or constrained state for delivery within the delivery sheath 12 (FIG. 1).

Each cuff 33 may have a width (i.e. in a longitudinal direction relative to the central axis of the device 10a) of approximately 0.0005 to 0.0015 inches, with the width of the gaps 31 (i.e. the longitudinal spacing between the cuffs 33) being in the range of 0.002 to 0.020 inches. The central portion 28 has a length in the range of 6-30 mm.

As shown in FIG. 3A, elongate standards or uprights 32 extend from the proximal portion 26 to the distal portion 30. The standards 32 provide axial strength to the central portion and aid in maintaining the desired spacing of the gaps 31. The standards may also be used to provide axial force to the device if it is necessary to re-position the device after a partial deployment within the vessel as discussed above.

In one embodiment, 2-8 standards may be used. Legs 34a extend from the proximal ends of a plurality of the standards 32, and legs 34b extend from the distal ends of a plurality of the standards 32. In the shown embodiment, legs 34a and legs 34b are on alternating ones of the standards, although other configurations may be used. Each of the legs 34a, 34b includes an eyelet 35a, 35b.

At the proximal portion 26, generally V-shaped strut members 36 are coupled between standards 32, with the apexes 38 of the strut members extending towards the central portion 28. At the distal portion 30, generally V-shaped strut members 40 are coupled between the standards 32, with the apexes 42 of the strut members 40 extending away from the central portion 28. Strut members 36, 40 help to maintain the cylindrical shape of the device 10a, and also facilitate collapsing of the device for loading of the device into the sheath 12 (FIGS. 2A and 2B) by providing folding points for the device. To fold the device for insertion into the sheath, a thread or wire is passed through eyelets 35b on legs 34b, and a second thread/wire is passed through the eyelets 35a of legs 34a. Tension is applied to the threads, thus pulling the legs 34a, 34b in the directions indicated by arrows in FIG. 3A, causing the device to fold along the apexes of the strut members 36, 40 and to thus place the device in a radially compressed configuration.

Loading the device 10a into the sheath is facilitated by the use of a funnel having its tapered end inserted into the distal end of the sheath. To load the device 10a into the sheath, the thread/wire passed through the eyelets 35a at the proximal end of the device is inserted into the flared end of funnel and through the sheath until it exits the proximal end of the sheath. Tension is applied to the threads at the proximal and distal ends of the device to fold the device 10a as discussed in the previous paragraph. The folded device is drawn through the funnel and into the sheath. The folding step is aided by passage of the device into the funnel.

FIG. 3B shows a second embodiment of an occlusion device 10b. In the device 10b, a plurality of helically-oriented bands 42 form the high coverage central region 28b. Bands 42 are preferably closely spaced to provide high coverage in the region 28b (e.g. 40-50% coverage). The distal ends of the bands 42 are connected to eight corresponding uprights 32 at the distal region 30b of the device, (although the device may include other numbers of uprights as discussed elsewhere). V-shaped struts 40b are coupled at their legs to the uprights 32. The proximal ends of the bands are connected to the apexes of V-shaped struts 36b at the proximal portion 26b of the device. Undulating cuff structures 44 intersect with the bands 42 and encircle the device as shown. These cuff structures 44 help prevent the device from flattening when positioned in or moved through bends in the vasculature. Legs 34a, 34b and eyelets 35a, 35b are provided as described above.

In an alternative device 10c shown in FIG. 3C, rather than having v-shaped struts 36, 40, the device 10c includes circumferential cuff members 46 extending between the standards 32a, 32b. Cuff members 46 include proximally oriented curves 48 near the point of intersection with standard 32a, and distally oriented curves 50 near the point of intersection with the standard 32b. As with the FIG. 3A embodiment, high coverage central portion 28c of the device is formed of closely spaced bands 33c of material. These bands 33c have slight curves 52, 54 near the standards, giving the bands 33c an identical or similar shape to the cuff members 54. As will be discussed below, these curves form fold points along which the device folds for insertion into the sheath 12 (FIG. 2A).

Standards 32a, 32b may include flexures such as s-curves 60 to add flexibility without significantly compromising column strength. As shown in FIG. 3D, additional flexures 60a on the standards 32 may be positioned between rows of the bands 33d within the high coverage central portion 28d to improve the kink resistance of the device. In this embodiment, the bands 33d have an undulating shape to accommodate the flexures 60a. Cuffs 46 may have a similar shape as shown.

Referring again to FIG. 3C, the steps of folding of the device 10c and inserting it into the sheath are performed in a manner similar to that described above. As illustrated by arrows A1 and A2, standard 32b is pulled in a distal direction while standard 32a is simultaneously pulled in a proximal direction. As the device 10c folds, the cuff members 46 fold at the curves 48, 50. Because the additional embodiments disclosed in this application are inserted into the sheath using a similar procedure, this procedure will not be repeated again in this disclosure.

As is evident from the figures, the standards 32 may have various configurations. Some standards may extent the length of the device (e.g. FIGS. 3C and 3D), while others may be only at a proximal or distal portion of the device (FIG. 3B). In other embodiments, standards may extend from the proximal or distal end of the device, through the high coverage central portion, and then terminate at a location short of the opposite end of the device. Any number of standards may be used, but between 2 and 8 standards are preferred. Standards 32a may be generally vertical as shown in FIGS. 3C and 3D or the device may use helical standards 32e as shown in FIG. 3E. In some embodiments, additional eyelets 35d may be included, such as on portions of the standards that are more central relative to the ends of the device as shown in FIG. 3D. During loading of the device, threads may be passed through these eyelets and used to compress the device for loading into the deployment sheath. The eyelets (as with the other eyelets described elsewhere herein) may also be include radiopaque marker material on them to aid in fluoroscopic positioning the device during implantation.

Another embodiment of a device offering very high coverage in the central area 28f is shown in FIG. 3F. Here, bands 33f are formed as wide bands having narrow slots 58.

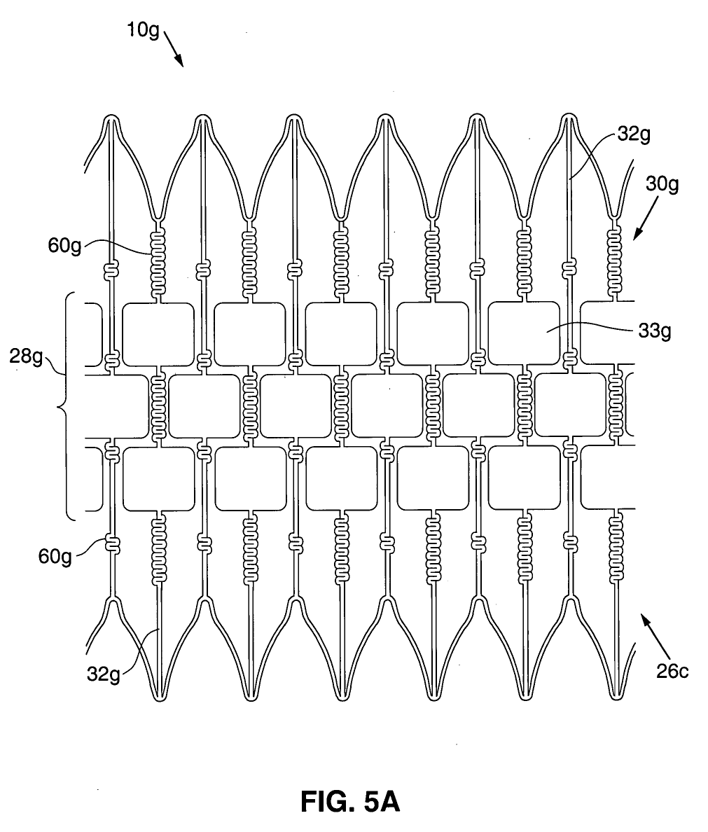

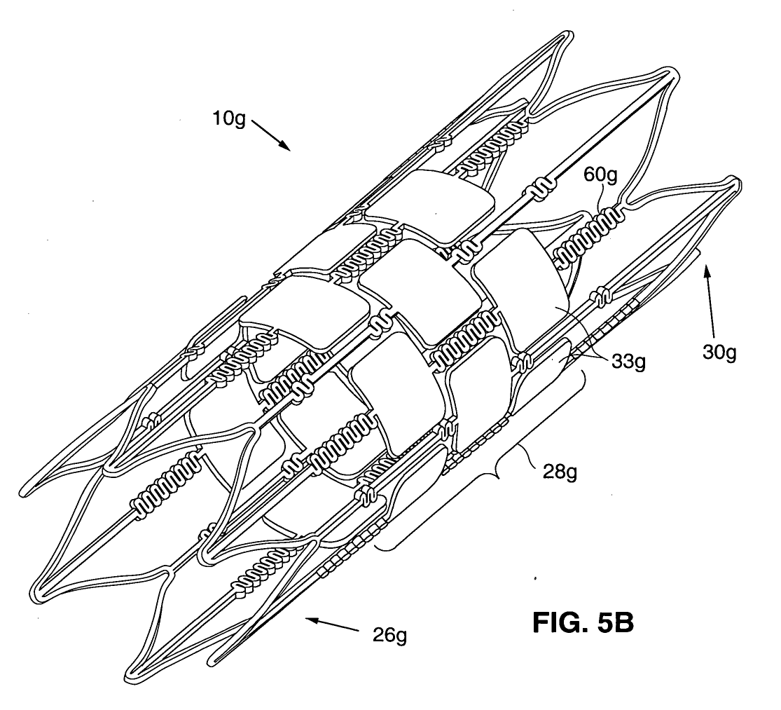

FIGS. 5A and 5B show an alternative embodiment of an occlusion device 10g. The FIG. 5A embodiment differs from the FIG. 3A-3F embodiments in that the high coverage central portion 28g of the device is formed of a plurality of paddles 33g. Paddles 33g are supported by standards 32g, which may include meandering flexures such as “S” shaped regions 60g. These paddles may be laser cut from the same tube, or formed using a different material such as PTFE or other polymers and attached to the standards 32g.

The device 10g is structured such that when some of the paddles 33g are positioned over a branch vessel, those paddles will be deflected outwardly by fluid pressure from the parent vessel to the branch vessel, thus allowing normal flow into the branch vessel to continue. However, those of the paddles 33g that are positioned over the aneurysm will have zero to limited deflection given the lack of a pressure differential between the parent vessel and the branch vessel, and will thus prevent the flow of blood into the aneurysm.

In a modification to the FIG. 5A embodiment shown in FIG. 6, occlusion device 10h may include a higher density arrangement of paddles 33h. Paddles may be supported by lateral struts 64 extending from the standards 32h. As shown, struts may include flexures having “S” patterns to permit deflection of the paddles as described in connection with the FIG. 5A embodiment. Paddles 33h may include perforations 66, or they may be provided without perforations (see paddles 33h′) for maximum coverage.

The FIG. 6 embodiment illustrates that additional support features may be included to the device if desired for structural rigidity or to facilitate loading of the device into the sheath 12 (FIG. 2A). For example, multiple rows of strut members 62 may extend between the standards 32h. Alternatively, or in addition to strut members 62, circumferential cuffs 68 may extend between the standards 32h. Standards 32h may include flexures 60h s-curves 52 to add flexibility without significantly compromising column strength.

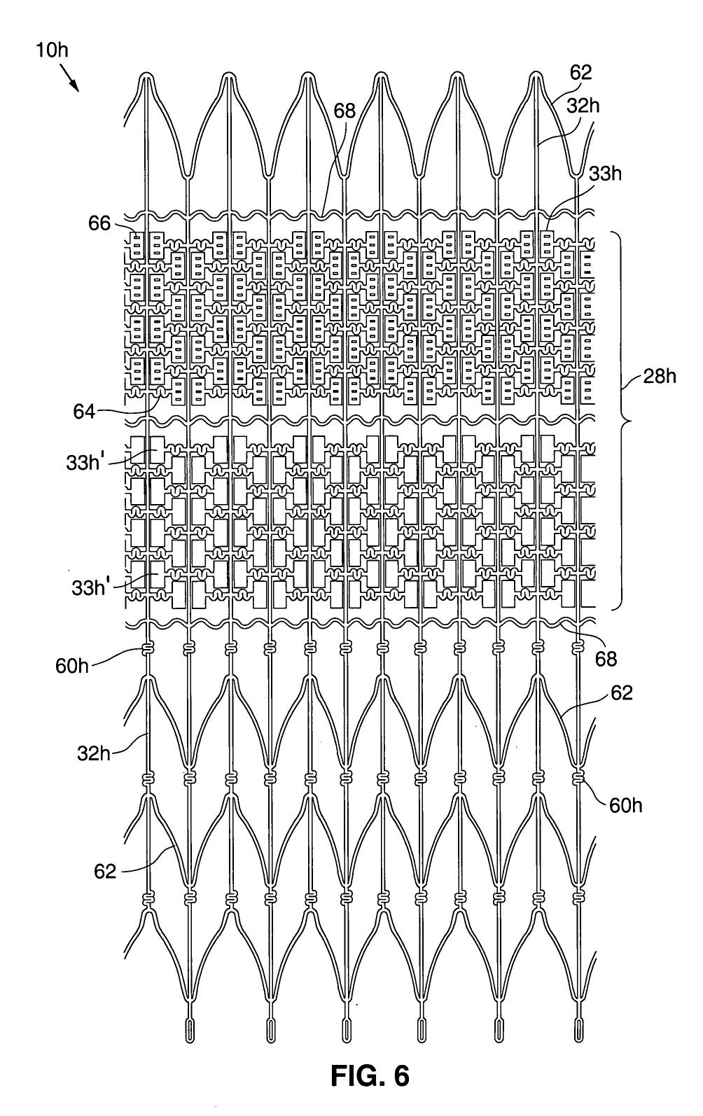

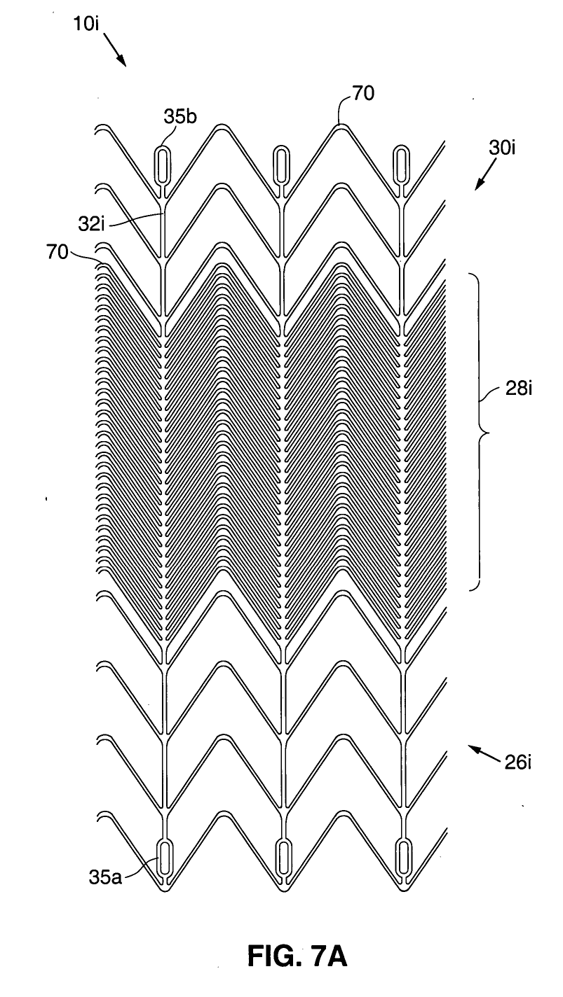

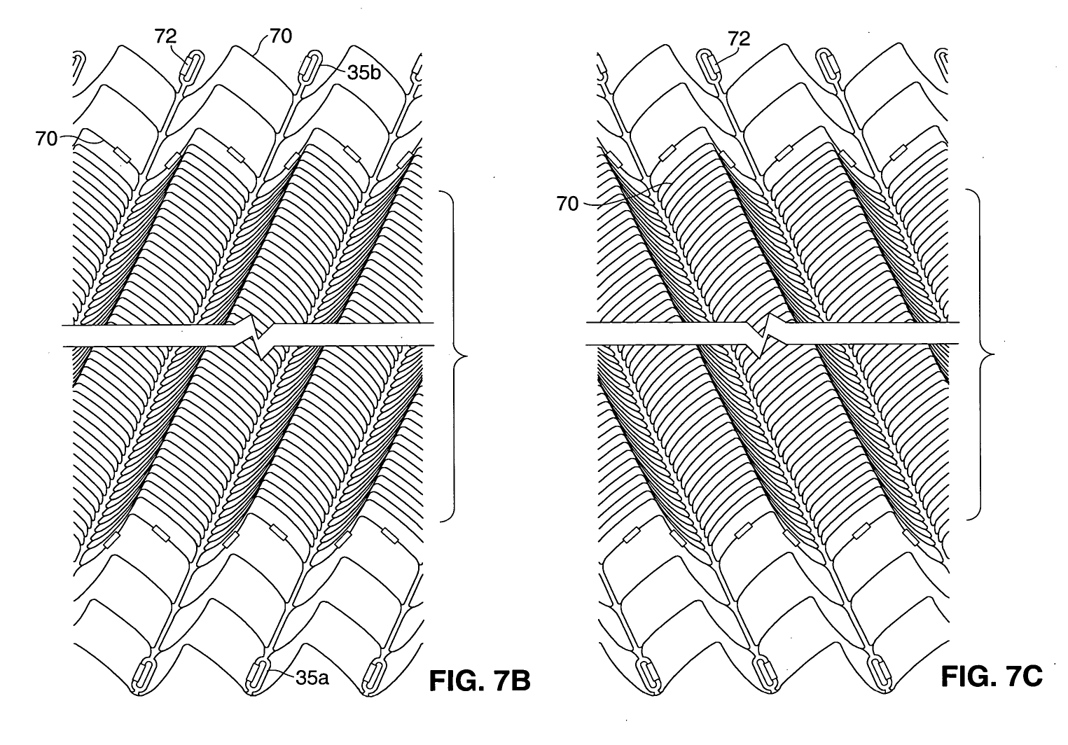

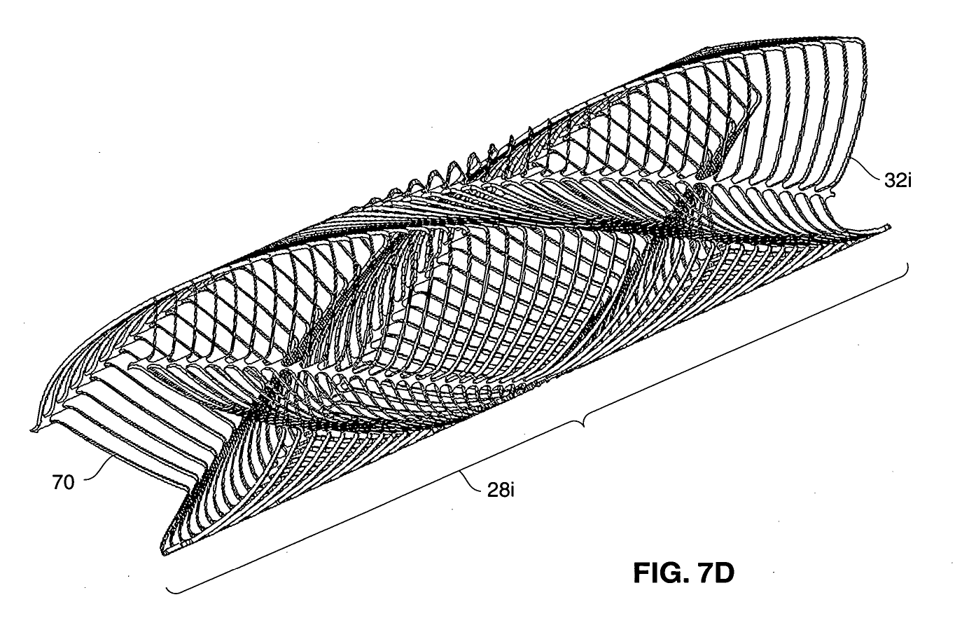

FIGS. 7A-7D illustrate another embodiment 10i of an implant. Referring to FIG. 7A, device 10i includes three uprights 321 coupled together by a plurality of V-shaped bands or connectors 70. This arrangement, as well as many of the others described herein, is beneficial in that the device does not significantly shorten in length as it expands from its radially compressed position when deployed within a vessel. During implantation, the physician first positions the device (compressed within the sheath 12) adjacent to an aneurysm neck A, and s/he then releases the device from the sheath. The term “foreshortening” is known in the art to refer to the amount by which the device shortens from the length it assumes within the catheter to the length it assumes when expanded into contact with the walls of the largest vessel for which the device is recommended. Significant foreshortening presents challenges to the physician, since it can cause a device that was aligned with the aneurysm when within the deployment sheath to shorten out of alignment with the aneurysm when it is released from the sheath. The present designs limit the amount of foreshortening to no more than 15%, and preferably to no more than 10%.

In the high coverage area, these V-shaped connectors 70 have a width (i.e. in a direction perpendicular to a long edge of the connector) of approximately 0.0005″-0.0012″. The gaps between the V-shaped connectors 70 have a width of approximately 0.005-0.015″ in a direction perpendicular to the long-edge of the V-shaped connectors 70. In the proximal and distal sections, the V-shaped connectors may have widths in the range of 0.0008″-0.0016″.

As shown, the V-shaped connectors are closely spaced in the high coverage area 281, and less closely spaced in the proximal and distal sections 261, 301. The apexes of the V-shaped connectors are pointed in a common direction to assist in loading of the device into the deployment sheath, and to allow the device to be withdrawn into the sheath if repositioning is needed during deployment. The proximal section 261 of the device maybe be provided to include additional length (compared with the length of the distal section) to allow the device to be resheathed during deployment if it becomes necessary. Thus, device 101 may be configured to have a proximal section 261 of 2-15 mm in length (preferably 3-7 mm), a high coverage section 281 of approximately 2-40 mm in length (preferably within the range of 10-14 mm), and a distal section 301 of 2-15 mm in length (preferably 3-5 mm). The outer diameter of the device, when fully expanded, is approximately 1-10 mm, and preferably 3.5-5.5 mm.

In one configuration, the device 101 is laser cut into a nitinol tube, and is then twisted and shape set to helically position the uprights 321. It has been found that a helical arrangement helps the deployed device conform to the vessel walls, and it also improves the ability of the device to resist kinking. FIG. 7B illustrates the device as it would appear longitudinally cut and laid flat following shape setting using a right hand twist. FIG. 7C is a similar drawing of the device as it would appear following shape setting using a left hand twist. FIG. 7D is a perspective view of the high coverage section 281 of the FIG. 7B device following shape setting. As illustrated in FIGS. 7B and 7C, radiopaque markers 72 are positioned on the eyelets 35a, 35b and V-connectors 70 just distal and just proximal to the high coverage section 281.

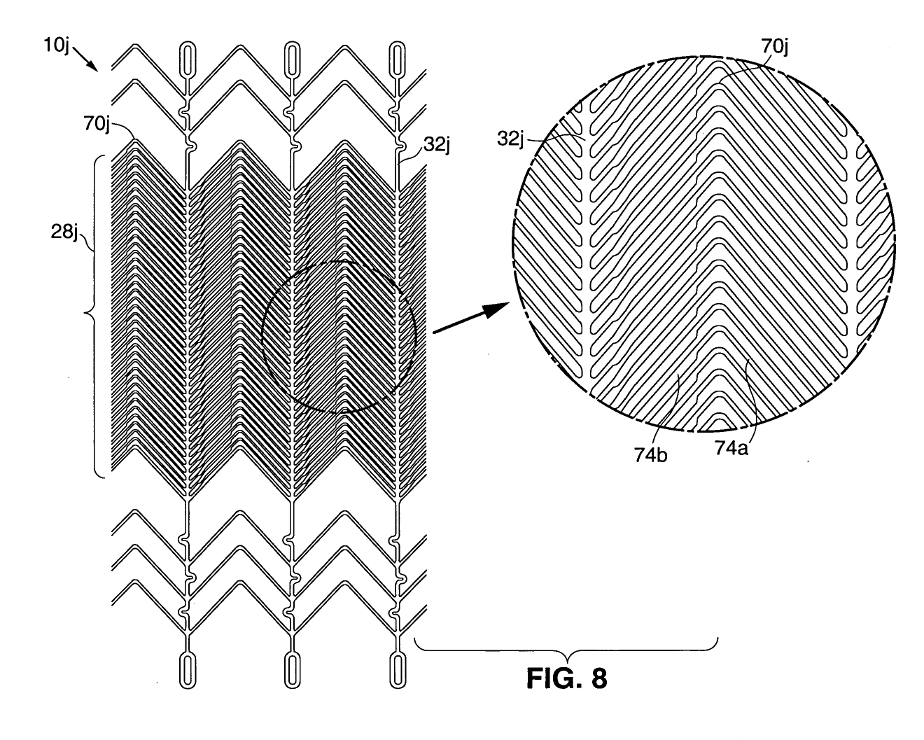

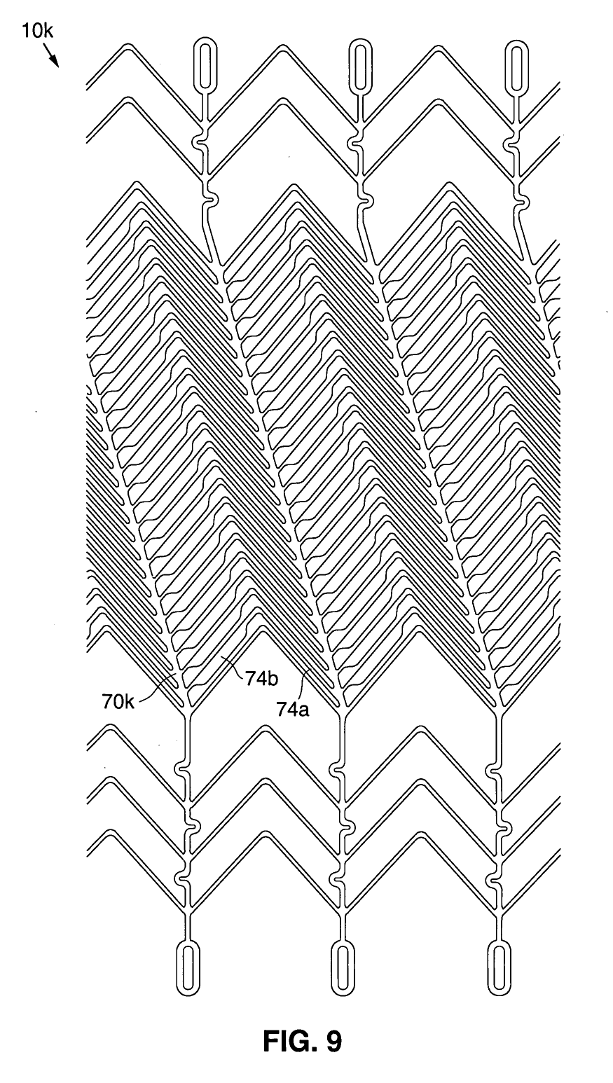

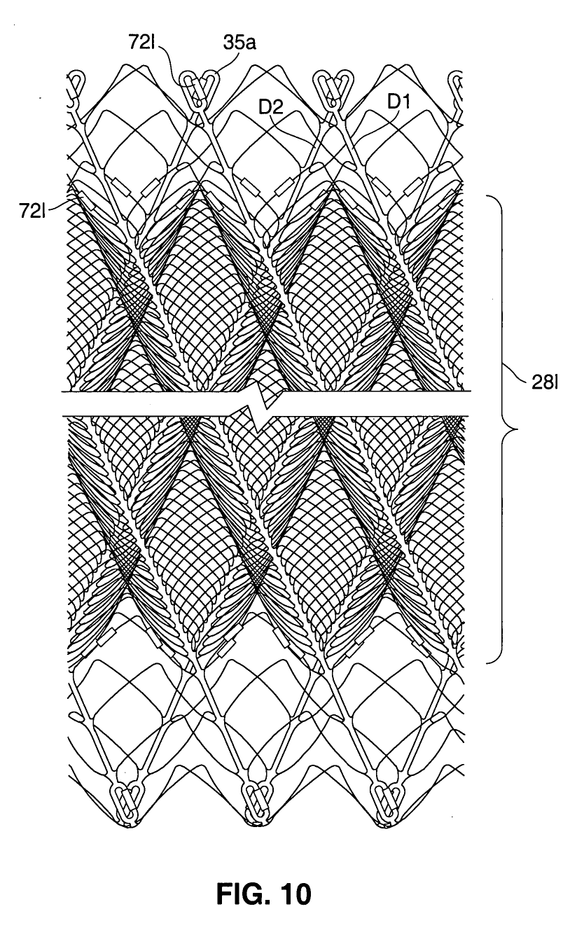

In some instances, shape setting the device 10i into a helix can result in the formation of gaps in the high coverage area 281 of the device. In particular, for any given one of the V-shaped connectors 70, forming the device into a helix will shift one leg of the “V” more closely to the corresponding legs of adjacent V-shaped connectors and will simultaneously enlarge the gap between the other leg of the “V” and the corresponding legs of adjacent V-shaped connectors. This can increase the blood flow into the aneurysm since it will decrease the percentage of metal covering some regions of the aneurysm while increasing the percentage of coverage over other regions. The device 10j shown in FIG. 8 is designed such that the widths of the gaps between the V-shaped connectors 70j will become uniform after the device is shape set into a helix. More specifically, the FIG. 8 embodiment is manufactured by cutting each “V” to include one narrow leg 74a and one broader leg 74b so that there initially is a larger gap between the legs 74a than between the legs 74b on adjacent connectors 70j. When the device is shape set, the change in shape of the V-shaped connectors 70j will cause the spacing between the legs 74a and the spacing between the legs 74b to be more or less equal. In a modified device 10k shown in FIG. 9, the device 10k is cut from the nitinol tube directly into a helical shape, again with one leg 74b of each V-shaped connector 70k having a larger width than the opposite leg 74a. In this embodiment, the shape setting step may be eliminated, or shape setting may be performed in order to increase the pitch angle of the helix.

Where it is desirable to further increase the percentage of coverage and reduce the pore size provided over the aneurysm, a pair of devices may be positioned within the vessel, with one device coaxially disposed within the other device. According to one embodiment, a first device having a left hand helical twist as shown in FIG. 7A is positioned within a vessel, bridging an aneurysm, and a second device having a right hand helical twist as shown in FIG. 7B is positioned within the first device, preferably with the high coverage central regions directly overlapping one another. The arrangement of the devices (if they were to be cut longitudinally and laid flat) is shown in FIG. 10, with one device labeled D1 and the other labeled D2. In this embodiment, the twist angle used to form the inner and outer devices is approximately 20-40 degrees.

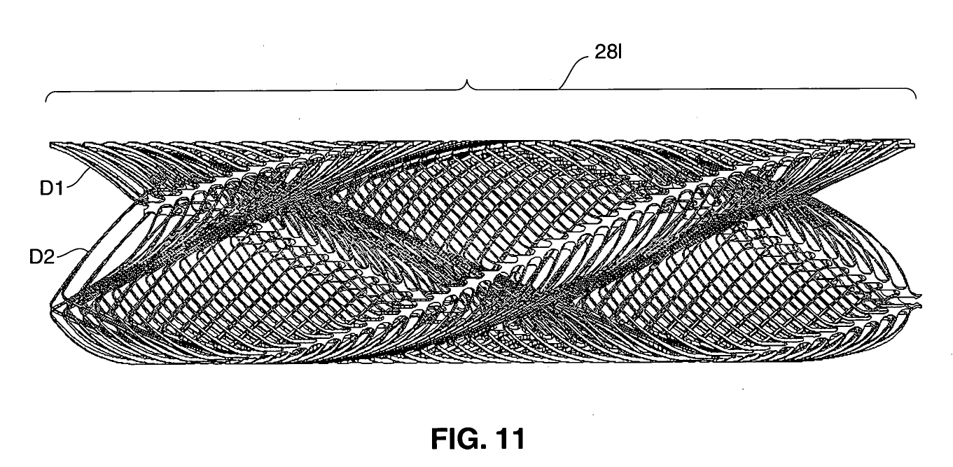

A perspective view of the high coverage section 281 of the nested devices is shown in FIG. 11. As can be seen, the combination of the two devices creates a mesh over the high coverage area 281, thus increasing the percentage of metal covering (and decreasing the pore size in the device at) the neck of the aneurysm. For example, taking as an example a device of the type shown in FIGS. 7A-7D, assuming for the purposes of the example the device has a gap between V-connectors of approximately 0.015″ in width and 0.110″ in length discussed, overlapping the device with an identical device having an opposed helical shape might position the V-connectors of the two devices to intersect to produce a combined pore size/gap size for the overlapping devices that is a 0.015″×0.015″ square rather than the 0.015″×0.110″ rectangle of a single device.

As shown in FIG. 10, radiopaque markers 721 on the eyelets ends of the device, and similar markers at the boundaries of the high coverage sections 281 allow the user to accurately align the devices under fluoroscopic visualization. In the disclosed embodiment, the devices need only be aligned longitudinally and not axially, thus avoiding the need to torque the deployment sheath and associated tools during implantation. Longitudinal alignment may be complete as shown in FIG. 10 or 11, or it may be partial.

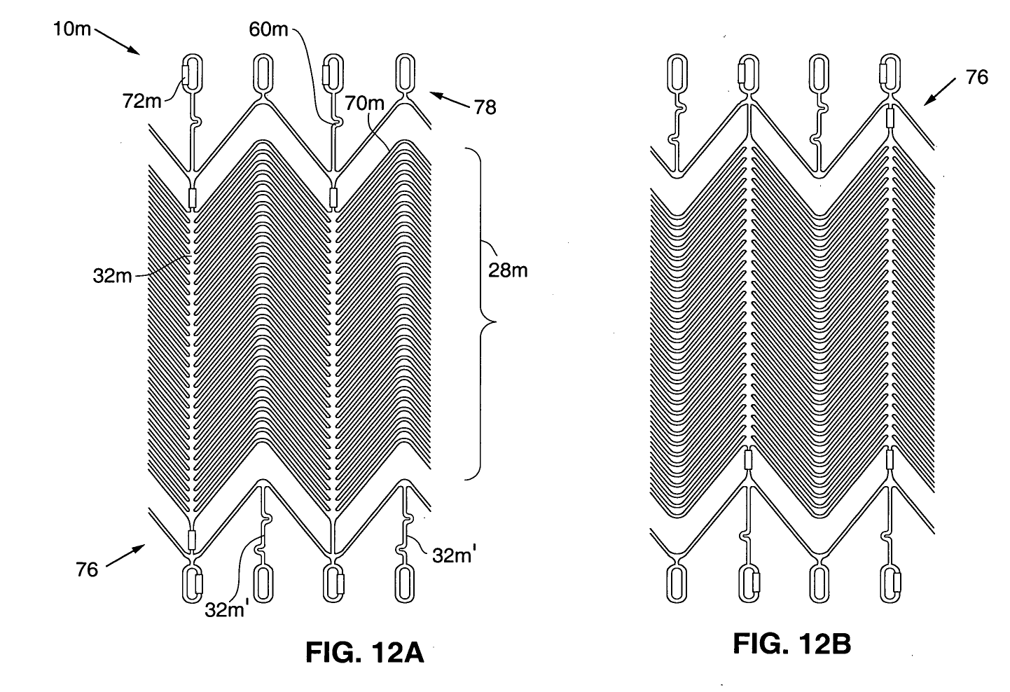

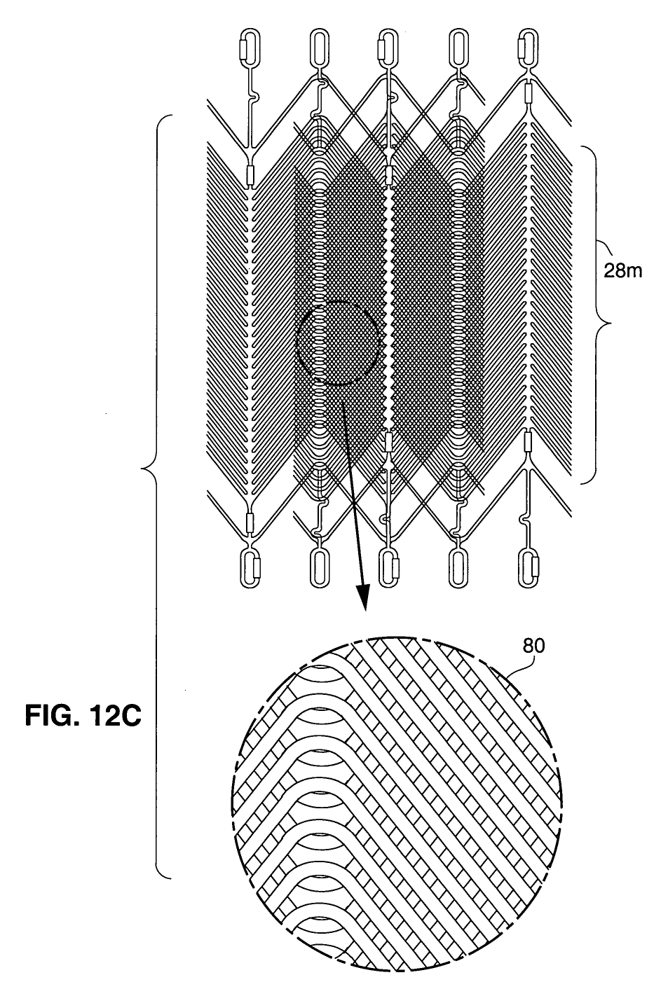

Non-helical devices may alternatively be deployed in an overlapping arrangement. FIG. 12A illustrates an embodiment of a device 10m that may be used for this purpose. As with the embodiments of FIGS. 7A-7C, the FIG. 12A embodiment includes V-shaped connectors 70m extending between uprights 32m. On a proximal end 76, a pair of additional uprights 32m′ is added at the apexes of the v-shaped connectors. On a distal end 78 shown at the top of the figure, additional eyelets are added. These extra features enhance the pushability of the device during deployment. As with previous designs, flexures 60m are positioned on the uprights 32m to allow the device to flex as it moves through the tortuous vasculature.

In one method of deploying the FIG. 12A embodiment, two identical devices are used. A first one of the devices is positioned within the vasculature with its high coverage region 28m positioned over the neck of an aneurysm, and then a second identical device is positioned within the first device with its high coverage region overlapping the high coverage region of the first device. For example, the first device might be positioned with end 76 oriented in a distal direction as shown in FIG. 12B, and the second device might be positioned with end 78 oriented in a distal direction as shown in FIG. 12A. This orientation for the second (inner) device is advantageous in that it positions the V-connectors 70m with the apexes pointing away from the deployment sheath, allowing the inner device to be resheathed if repositioning is needed during deployment. However, the devices can also be positioned such that the first (outer) device is in the resheathable orientation (the orientation shown in FIG. 12A) and the second (inner) device is in the orientation shown in FIG. 12B.

As shown in the enlarged section of FIG. 12C, by orienting the V-connectors 70m of the first and second devices in opposite directions, a mesh-type arrangement 80 is formed in the high coverage area 28m.

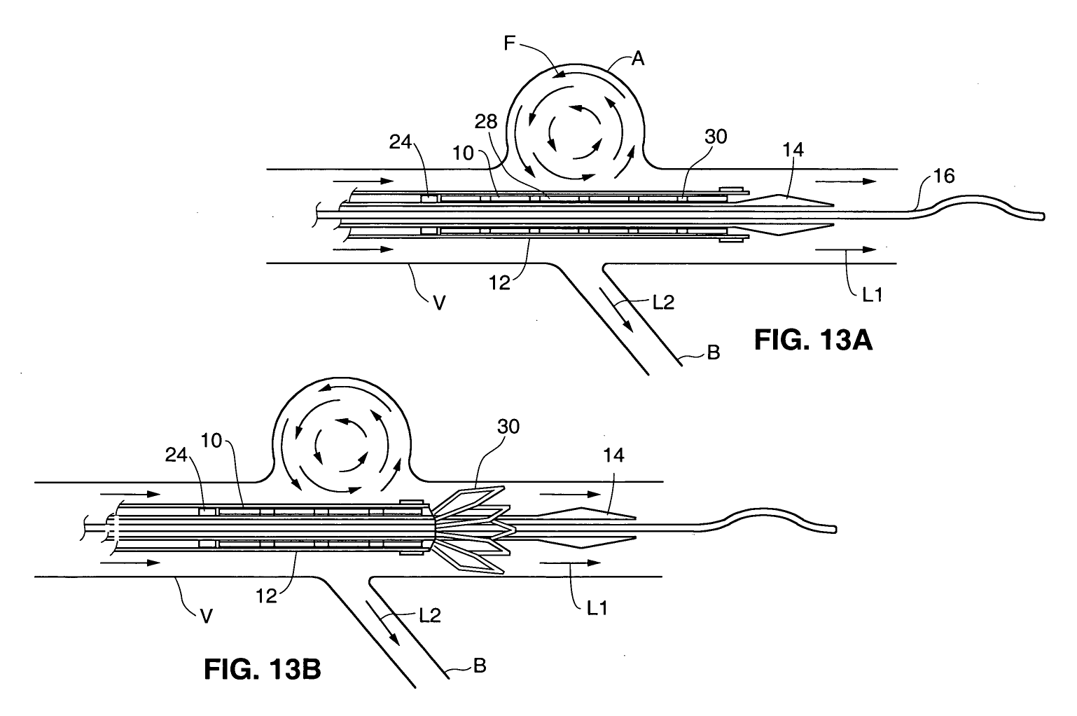

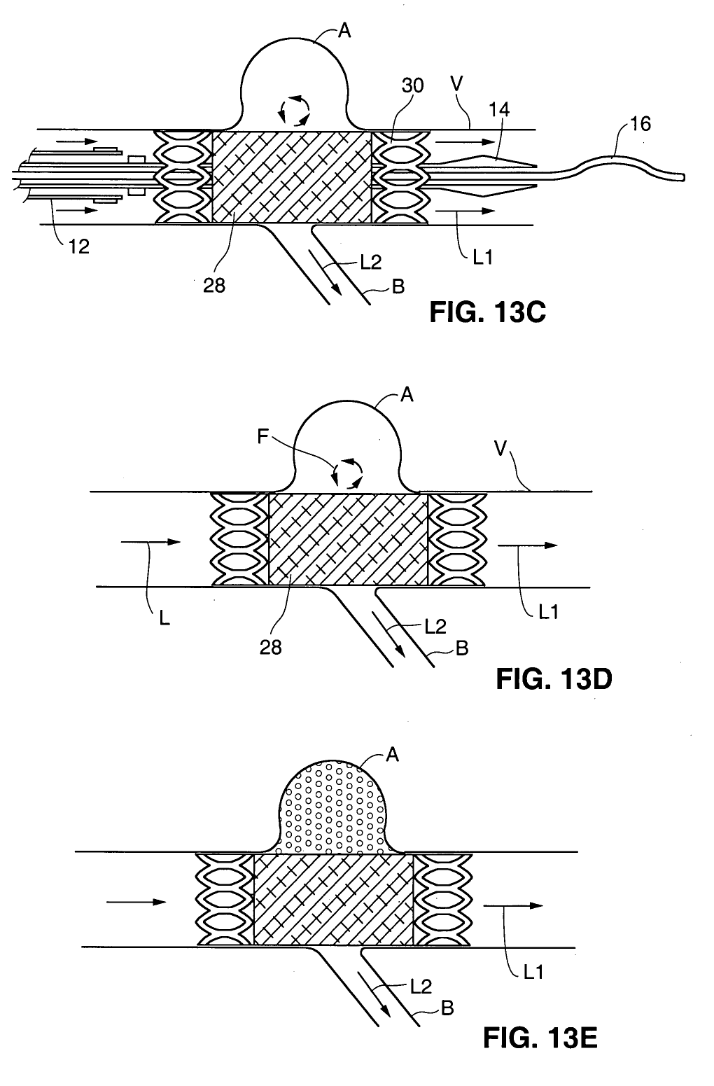

Deployment and use of the system will be described in connection with FIGS. 13A-13E. This description will be given in the context of an aneurysm A located in close proximity to a branch vessel B (see also FIG. 1). Vortex flow of blood within the aneurysm is represented by arrows F. Laminar flow of blood within the parent vessel and the branch vessel is represented by arrows L1 and L2, respectively.

Prior to use, the device 10, sheath 12 and pusher 14 are assembled as described in connection with FIG. 2B. Sheath 12 maintains the device 10 in the constrained position shown in FIG. 2B.

Referring to FIG. 13A, a guidewire 16 is introduced into the vasculature and advanced beyond the aneurysm A under fluoroscopic visualization. The pusher 14 (with the device 10 and sheath 12 thereon) is advanced over the guidewire until the distal portion 30 of the device is positioned beyond the aneurysm A and the central portion 28 of the device 10 is positioned adjacent to the aneurysm. The sheath 12 is then withdrawn as shown in FIG. 13B, causing the distal portion 30 to self-expand into contact with the wall of the vessel V, beyond the aneurysm. During retraction of the sheath 12, pressure is maintained against the proximal end of pusher 14 so that shoulder 24 of the sheath holds the device at the target deployment site.

Continued retraction of the sheath 12 causes the central portion 28 of the device 10 to be deployed adjacent to the aneurysm (FIG. 13C). Once the device 10 is deployed, the sheath, pusher and guidewire are withdrawn from the body. As shown in FIG. 13D, the presence of the device 10 diminishes blood flow into the aneurysm, causing the vortex flow F within the aneurysm to taper off. The aneurysm A eventually clots off, forms a scar, and heals as represented in FIG. 13E. As discussed above, laminar flow L2 through branch vessel B continues and is relatively unimpeded by the presence of the device 10.

The system 100 (FIG. 2A) is preferably packaged with instructions for use setting forth the steps for deploying the occlusion device, as well as for resheathing and/or repositioning the device as needed.

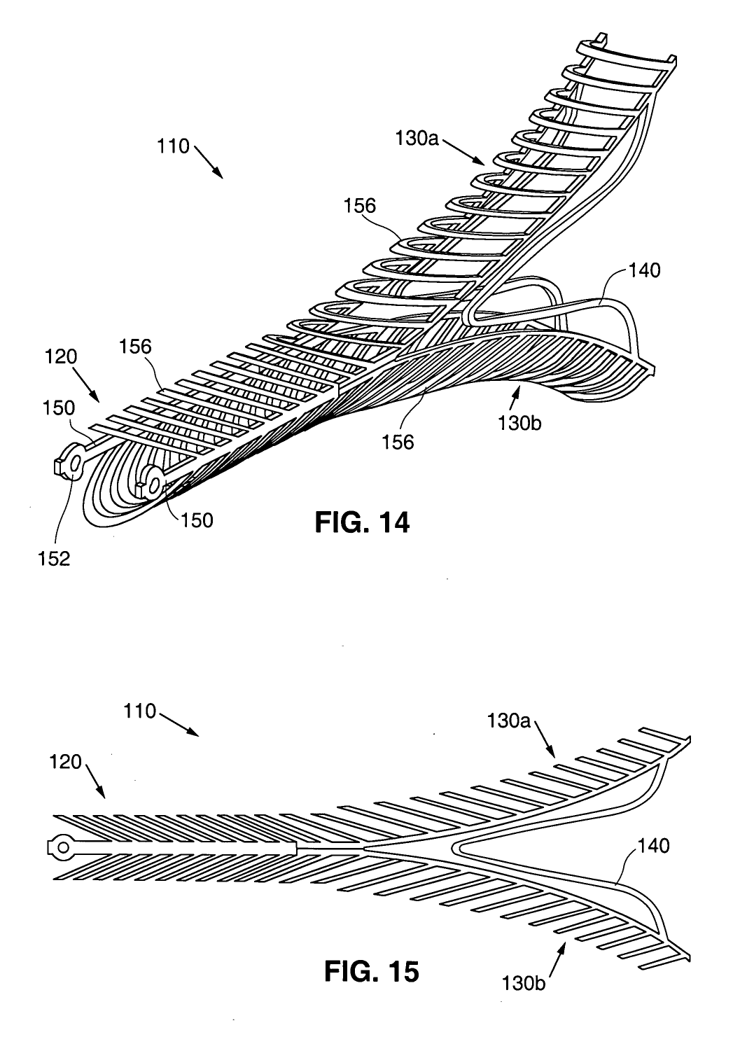

The devices described above are particularly useful for providing occlusion at the neck of an aneurysm located along a single blood vessel. At times, however, aneurysms will appear at a vessel bifurcation at the point of bifurcation. FIGS. 14 and 15 illustrate an aneurysm occlusion device that will occlude this type of aneurysm using a single device while maintaining an undisturbed blood flow through the parent vessel bifurcation. The device is designed such that when it is deployed within a bifurcated vessel, the high coverage portion of the device will be positioned at the neck of the aneurysm for optimal occlusion.

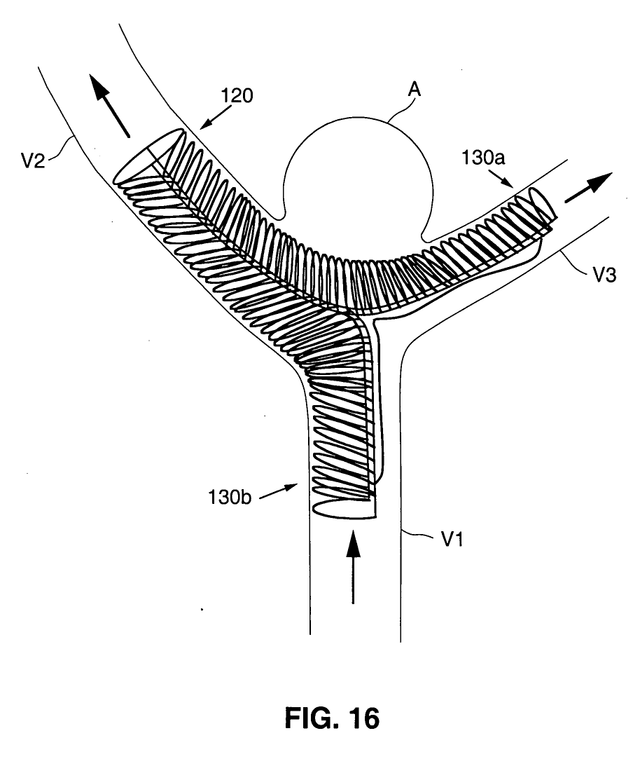

Referring to FIG. 15, occlusion device 110 is a generally Y-shaped device having a distal stem portion 120 and a pair of proximal branches 130a, 130b. A pair of V-members 140 extend between the proximal branches 130a, 130b. V-members 140 are foldable at their apexes to allow the proximal branches 130a, 130b to be brought close together for positioning of the device within a deployment catheter prior to implantation. Once released from the catheter, the V-members 140 return to the position shown in FIGS. 14 and 15 to restore the Y-shape of the device 110, such that each of the branches 130a, 130b and the stem portion 120 may be disposed within a separate branch of a bifurcation (FIG. 16). The V-members gently press the branches 130a, 130b against the walls of the corresponding vessels to anchor the device in place.

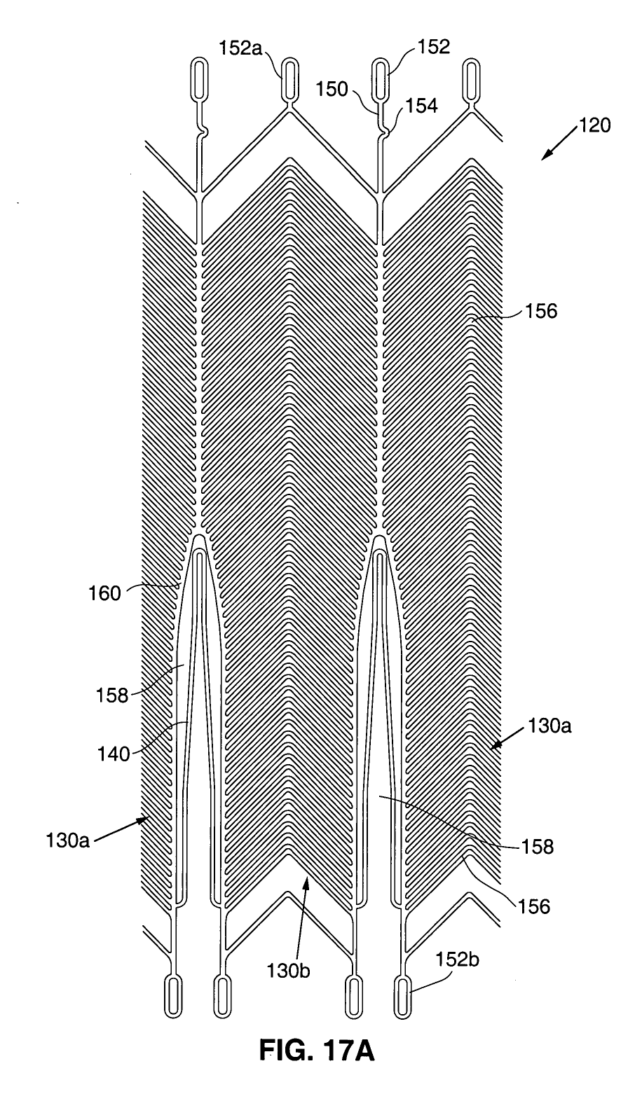

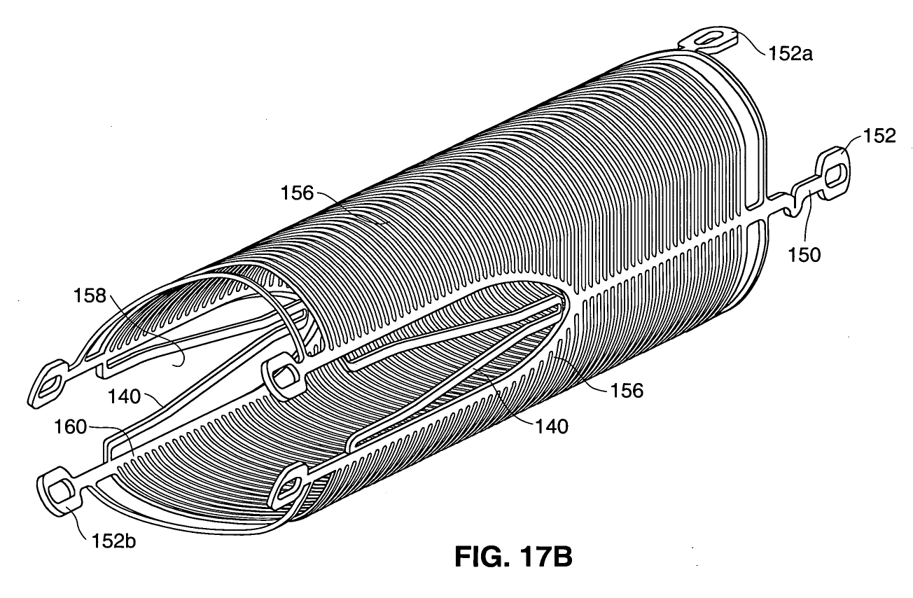

In one method of manufacturing, the device 110 is laser cut from Nitinol alloy tubing. FIG. 17A shows a flat view of the pattern into which the tubing might be cut to form the device. The pattern is shown flat to clearly show the detail. Thus, the branch 130a is shown in two pieces on the left and right hand sides of the drawing even though the branch 130a is a single component cut along the cylindrical tubing.

Device 110 includes a pair of uprights 150 extending from the distal end. Uprights might include eyelets 152 and flexures 154 as discussed with prior embodiments. V-shaped connectors 156 extend between the uprights. Additional eyelets 152a may be coupled to the apexes of the connectors 156 at the distal end of the device.

Towards the proximal end of the device, the circumferential length of the v-shaped connectors 156 decreases to create spaces 158 between the branches 130a, 130b. Each of the uprights 150 forms a fork having legs 160 bordering the spaces 158. V-members 140 are connected to the legs 160 and oriented with their apexes within the spaces 158 as shown. Eyelets 152b are positioned on the proximal ends of the legs 160.

In one method of making the device, the tubing is cut according to this or a similar pattern, and then shape set to separate the branches 130a, 130b into the position shown in FIGS. 14-16.

In one embodiment, the device is formed of a nitinol tube having a wall thickness of approximately 0.001″ to 0.007″, the uprights have a width in the range of 0.001″ to 0.007″, the v-shaped connectors 156 forming the high coverage area of the device have a width of 0.0005″-0.002″, and the width of the V-members 140 is approximately 0.001″-0.005″. These dimensions are given by way of example only, as devices may be made according to a number of different dimensions. As with the other embodiments described above, the device 110 (FIG. 15) preferably includes radiopaque markers to allow implantation under fluoroscopic visualization.

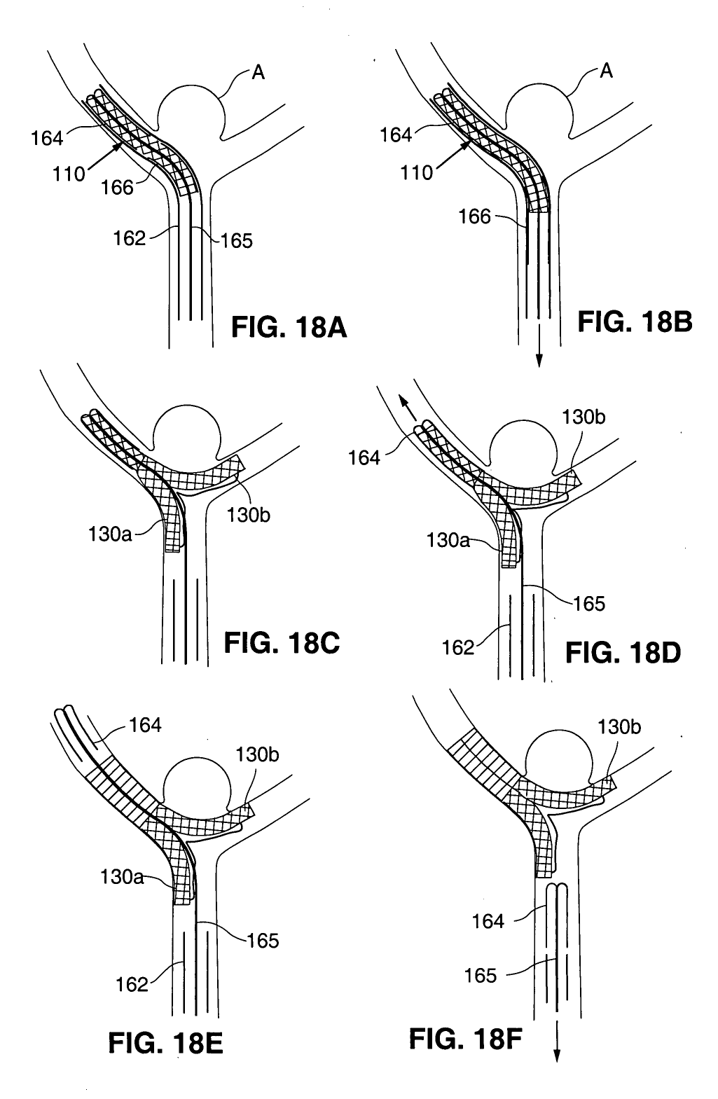

FIGS. 18A-18F illustrate one deployment sequence that may be used to deploy the device 110 over an aneurysm located at a bifurcation comprised of vessel branches V1, V2, and V3. As shown, the device 110 is positioned on a delivery catheter 162. The distal portion 120 is compressed by a distal sheath 164. A mandrel 165 extending through the distal portion 120 is coupled to the distal end of the sheath 164. The bifurcated proximal branches 130a, 130b are restrained within a proximal sheath 166.

With the distal portion 120 of the device in vessel V2, the proximal sheath 166 is pulled proximally (FIG. 18B), causing the proximal branches 130a, 130b of the device to be released and to expand to their open shape-set position (FIG. 18C). The catheter is manipulated using back and forth movement to position one of the proximal branches 130a bridging the neck of the aneurysm A and extending towards vessel V3, and to position the other branch within vessel V1. The distal sheath 164 is pushed distally using the mandrel 165 (FIG. 18D) to release the distal portion 120 of the device 110 (FIG. 18E). The distal sheath 164 is withdrawn through the lumen of the device, and the delivery system is removed from the body. Although this deployment method is described in connection with the bifurcated device, it may also be used to deploy any of the other devices disclosed devices, or devices outside the field of aneurysm occlusion (e.g. stents) in a distal-end-first manner.

Any of the features described in this application may be combined with each other and with other features in a variety of ways without exceeding the scope of the invention.

It should be recognized that a number of variations of the above-identified embodiments will be obvious to one of ordinary skill in the art in view of the foregoing description. Accordingly, the invention is not to be limited by those specific embodiments and methods of the present invention shown and described herein. Rather, the scope of the invention is to be defined by the claims and their equivalents.

We claim:

1. An aneurysm occlusion device positionable within a cerebral blood vessel covering a neck of an aneurysm on the blood vessel, the device comprising:
a tubular element having a lumen, the tubular element including an occlusive sidewall including a plurality of gaps, the gaps of a size sufficiently small to cause at least partial occlusion against flow of blood from the blood vessel through the side wall into the aneurysm, wherein the gaps are proportioned to allow flow of fluid through the side wall between the blood vessel and a side branch vessel in response to a fluid pressure differential between a first area inside the lumen and a second area outside the lumen.

2. The occlusion device of claim 1, wherein the gaps are expandable in response to the fluid pressure differential to allow flow of fluid through the side wall.

3. The occlusion device of claim 1, wherein the tubular element includes a plurality of bands having the gaps between them, and at least two elongate members extending from a proximal portion of the sidewall to a distal portion of the sidewall, each band including at least one end connected to one of the elongate members.

4. The occlusion device of claim 1, wherein each band includes a first end connected to a first one of the elongate members and a second end connected to a second one of the elongate members.

5. The occlusion device of claim 2, wherein the bands are deflectable in response to the fluid pressure differential to expand the gaps.

6. The occlusion device of claim 1, wherein the bands are parallel to one another.

7. The occlusion device of claim 1, wherein the elongate members extend longitudinally from a proximal portion of the sidewall to a distal portion of the sidewall.

8. The occlusion device of claim 1, wherein the elongate members include flexures.

9. The occlusion device of claim 1, wherein the tubular element includes between 2-8 elongate members.

10. The occlusion device of claim 1, wherein the elongate members extend helically from the proximal portion to the distal portion.

11. The occlusion device of claim 1, wherein the bands include a first portion having a first width, and second portion having a second width, wherein the first width is greater than the second width.

12. The occlusion device of claim 1, wherein the bands are v-shaped bands having an apex, the apex extending in a longitudinal direction.

13. The occlusion device of claim 11, wherein the bands have a first leg and a second joined at the apex, and wherein the first leg is wider than the second leg.

14. The occlusion device of claim 1, wherein the tubular element is an outer tubular element and wherein the device further includes an inner tubular element having a second occlusive sidewall, the inner tubular element positionable within the lumen of the outer tubular element with the second occlusive sidewall overlapping the sidewall of the outer tubular element.

15. The occlusion device of claim 14, wherein the second occlusive sidewall includes a plurality of second bands and at least two second elongate members extending from a proximal portion of the second sidewall to a distal portion of the second sidewall, each second band including at least one end connected to one of the second elongate members.

16. The occlusion device of claim 15, wherein the elongate members of the outer tubular element extend helically in a first direction, and wherein the second elongate members of the inner tubular element extending helically in a second direction opposite from the first direction.

17. The occlusive device of claim 16, wherein the first direction is clockwise and the second direction is counterclockwise.

18. The occlusive device of claim 16, wherein the first direction is counterclockwise and the first direction is clockwise.

19. The occlusive device of claim 1, wherein the occlusive device is functionally uniform around the circumference of the occlusive sidewall.

20. The occlusion device of claim 1, wherein the tubular member is radially expandable from a compressed position within a sheath to an expanded position within a blood vessel, and wherein the tubular member has a length in the compressed position that is longer than the length in the expanded position by an amount less than or equal to 15%.

21. The occlusive device of claim 1, wherein the tubular member is proportioned to be compressible to a diameter suitable for insertion into a microcatheter having an inner diameter of 2 mm or less.

22. The occlusion device of claim 1, wherein the tubular element includes a proximal section positioned proximally of the occlusive sidewall portion, and a distal section position distally of the occlusive sidewall portion, the proximal and distal sections including sidewalls that are less occlusive to blood flow than the occlusive sidewall portion.

23. The occlusion device of claim 1, wherein the sidewalls are non-braided and non-woven.

24. The occlusive device of claim 14, wherein the bands and the elongate elements are cut from a length of tubing.

25. The occlusive device of claim 1, wherein the tubular member includes a first end positionable in a first blood vessel and a bifurcated second end having bifurcated sections positionable in second and third vessels.

26. A method of treating an aneurysm in a blood vessel, comprising the steps of:
introducing into the blood vessel a tubular element having an occlusive sidewall, the sidewall defining a lumen and including a plurality of gaps,
covering a neck of the aneurysm with the occlusive sidewall, wherein the tubular element substantially occludes flow of blood through the sidewall into the aneurysm, and wherein the gaps, in response to a fluid pressure differential between a first area inside the lumen and a second area outside the lumen, allow flow of fluid through the side wall between the blood vessel and a side branch vessel.

27. The method according to claim 26, wherein the gaps expand in response to a fluid pressure differential between the first area and the second area.

28. The method of claim 27, wherein occlusion device of claim 1 wherein the tubular element includes a plurality of bands having the gaps between them, and wherein the bands deflect in response to the fluid differential to expand the gaps.

29. The method of claim 26, wherein the tubular element is an outer tubular element and wherein the method further includes positioning an inner tubular element having a second occlusive sidewall within the lumen of the outer tubular element.

30. The method of claim 29, wherein the inner tubular element is introduced into the lumen after the outer tubular element is introduced into the blood vessel.

31. The method of claim 29, wherein the inner tubular element includes first radiopaque markers, wherein the second tubular element includes second radiopaque markers, and wherein the method includes aligning the first and second markers under fluoroscopic visualization.

32. The method of claim 26, wherein the tubular element includes a first end and a bifurcated end having first and second bifurcations, and wherein the method includes positioning the first end in a first blood vessel, positioning the first bifurcation in a second blood vessel, and positioning the second bifurcation in a third blood vessel.

33. The method of claim 26, wherein the method includes radially compressing the tubular element, inserting the tubular element into a sheath, passing the sheath into the cerebral blood vessel, and releasing the tubular element from the sheath to cover the neck.

34. The method of claim 33, wherein the method includes fluroscopically observing release of the tubular element from the sheath.

35. The method of claim 34, wherein the method includes, during the releasing step, withdrawing the tubular element from the blood vessel into the sheath, repositioning the sheath, and releasing the tubular element from the sheath.

36. The method according to claim 33, wherein the method includes:
positioning a distal portion of the tubular element in the sheath, wherein the sheath is a distal sheath;
positioning the proximal portion of the tubular element in a second sheath;
passing the device with the sheaths thereon into the blood vessel;
removing the distal sheath to release the distal portion of the tubular element; and
removing the proximal sheath to release the proximal portion of the tubular element.

37. The method according to claim 36, wherein removing the distal sheath includes pushing the distal sheath in a distal direction, and wherein removing the proximal sheath includes withdrawing the proximal sheath in a proximal direction.

38. The method according to claim 36, wherein the step of removing the distal sheath is performed prior to the step of removing the proximal sheath.
